(19) United States
(12) Patent Application Publication
(10) Pub. No.: US 2011/0288599 A1
(43) Pub. Date: Nov. 24, 2011

(54) Bone Anchors

(76) Inventors: Michael Michielli, Raynham, MA (US); Bryan S. Jones, West Roxbury, MA (US); Philip Cormier, Raynham, MA (US); Hassan Serhan, Raynham, MA (US)

(21) Appl. No.: 13/110,378

(22) Filed: May 18, 2011

Related U.S. Application Data
(60) Provisional application No. 61/346,157, filed on May 19, 2010.

(51) Int. Cl. A61B 17/86

(52) U.S. Cl. 606/305

(57) ABSTRACT

A bone anchor assembly includes a bone anchor, a receiver member for receiving a spinal fixation element to be coupled to the bone anchor, and a closure mechanism to fix the spinal fixation element with respect to the receiver member. The bone anchor includes a distal shaft having a distal threaded section and a proximal threaded section. The distal threaded section has a first pitch and a first number of thread starts and the proximal threaded section has a second pitch less than the first pitch and a second number of thread starts greater than the first number of thread starts. The distal threaded section and the proximal threaded section have a constant lead.

### REFERENCE TO RELATED APPLICATIONS

This application claims the benefit of U.S. Provisional Application No. 61/346,157, filed May 19, 2010, which is incorporated herein by reference.

### BACKGROUND

Bone anchors may be used in orthopedic surgery to fix bone during the healing or fusion process. In spinal surgery, bone anchors may be used with spinal fixation elements, such as spinal rods, to stabilize multiple vertebrae either rigidly, in which no relative motion between the vertebrae is desired, and dynamically, in which limited, controlled motion between the vertebrae is desired. One problem with the use of bone anchors is that bone anchors may pullout or otherwise be displaced from the bone prior to the healing or fusion process completing. This problem is particularly common when a bone anchor is positioned in poor quality bone such as osteoporotic bone. Accordingly, there is need for improved bone anchors that minimize instances of anchor pull out.

### SUMMARY

Disclosed herein are improved bone anchor assemblies and, in particular, improved bone anchor assemblies used in connection with spinal fixation elements to fix multiple vertebrae either rigidly or dynamically.

In accordance with one aspect, a bone anchor assembly may include a bone anchor, a receiver member for receiving a spinal fixation element to be coupled to the bone anchor, and a closure mechanism to capture a spinal fixation element within the receiver member and fix the spinal fixation element with respect to the receiver member. The bone anchor may have a proximal head and a distal shaft configured to engage bone. The distal shaft may include a distal threaded section and a proximal threaded section. The distal threaded section may have a first pitch and a first number of thread starts and the proximal threaded section may have a second pitch less than the first pitch and a second number of thread starts greater than the first number of thread starts. The distal threaded section and the proximal threaded section may have a constant lead. The receiver member may have a proximal end having a pair of spaced apart arms defining a recess therebetween and a distal end having a distal end surface defining opening through which at least a portion of the bone anchor extends. The closure mechanism may be positionable between and may engage the receiver member to capture a spinal fixation element within the receiver member and fix the spinal fixation element with respect to the receiver member.

### BRIEF DESCRIPTION OF THE FIGURES

These and other features and advantages of the devices and methods disclosed herein will be more fully understood by reference to the following detailed description in conjunction with the attached drawings in which like reference numerals refer to like elements through the different views. The drawings illustrate principles of the devices and methods disclosed herein and, although not to scale, show relative dimensions.

FIG. 1 is a perspective view of an exemplary embodiment of a bone anchor assembly;

FIG. 2 is a side view of the bone anchor assembly of FIG. 1;

FIG. 3 is a side view in cross section of the bone anchor assembly of FIG. 1;

FIG. 4 is a side view of the bone anchor of the bone anchor assembly FIG. 1;

FIG. 5 is a cross sectional view of the distal threaded section of the bone anchor of the bone anchor assembly FIG. 1; and

FIG. 6 is a cross sectional view of the proximal threaded section of the bone anchor of the bone anchor assembly FIG. 1.

### DETAIL DESCRIPTION OF EXEMPLARY EMBODIMENTS

Certain exemplary embodiments will now be described to provide an overall understanding of the principles of the structure, function, manufacture, and use of the devices and methods disclosed herein. One or more examples of these embodiments are illustrated in the accompanying drawings. Those of ordinary skill in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The articles “a” and “an” are used herein to refer to one or to more than one (i.e. to at least one) of the grammatical object of the article. By way of example, “an element” means one element or more than one element.

The terms “comprise,” “include,” and “have,” and the derivatives thereof, are used herein interchangeably as comprehensive, open-ended terms. For example, use of “comprising,” “including,” or “having” means that whatever element is comprised, had, or included, is not the only element encompassed by the subject of the clause that contains the verb.

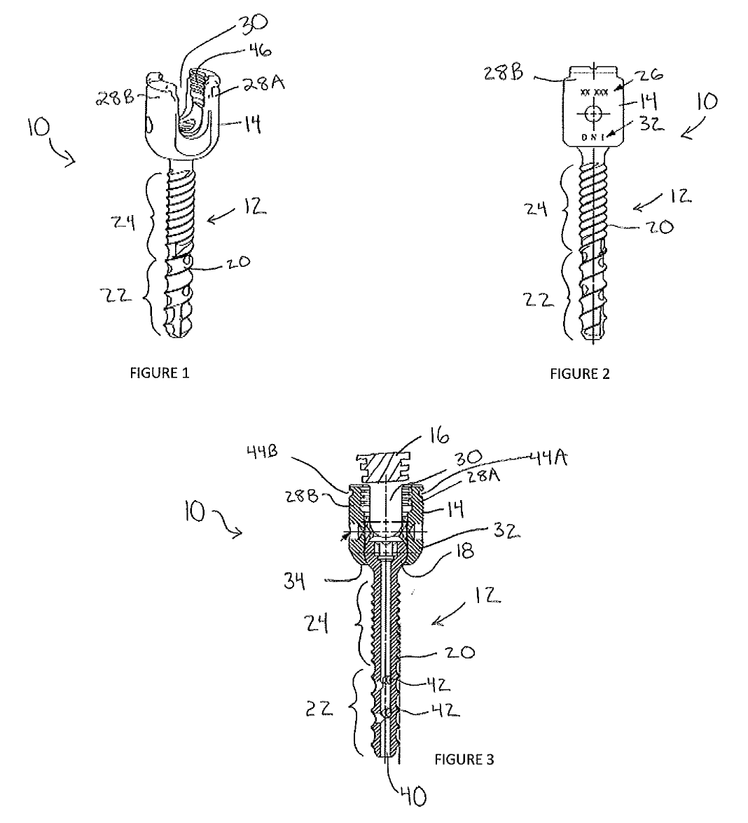

FIGS. 1-3 illustrate an exemplary embodiment of a bone anchor assembly 10 including a bone anchor 12, a receiver member 14 for receiving a spinal fixation element, such as a spinal rod, to be coupled to the bone anchor 12, and a closure mechanism 16 to capture a spinal fixation element within the receiver member 14 and fix the spinal fixation element with respect to the receiver member 14. The bone anchor 12 includes a proximal head 18 and a distal shaft 20 configured to engage bone. The distal shaft 20 has a distal threaded section 22 and a proximal threaded section 24. The distal threaded section 22 may have a first pitch and a first number of thread starts and the proximal threaded section 24 may have a second pitch less than the first pitch and a second number of thread starts greater than the first number of thread starts. The distal threaded section 22 and the proximal threaded section 24 may have a constant lead. The receiver member 14 has a proximal end 26 having a pair of spaced apart arms 28A, 28B defining a recess 30 therebetween and a distal end 32 having a distal end surface 34 defining opening through which at least a portion of the bone anchor 12 extends. The closure mechanism 16 may be positionable between and may engage the arms 28A, 28B to capture a spinal fixation element within the receiver member 14 and fix the spinal fixation element with respect to the receiver member 14.

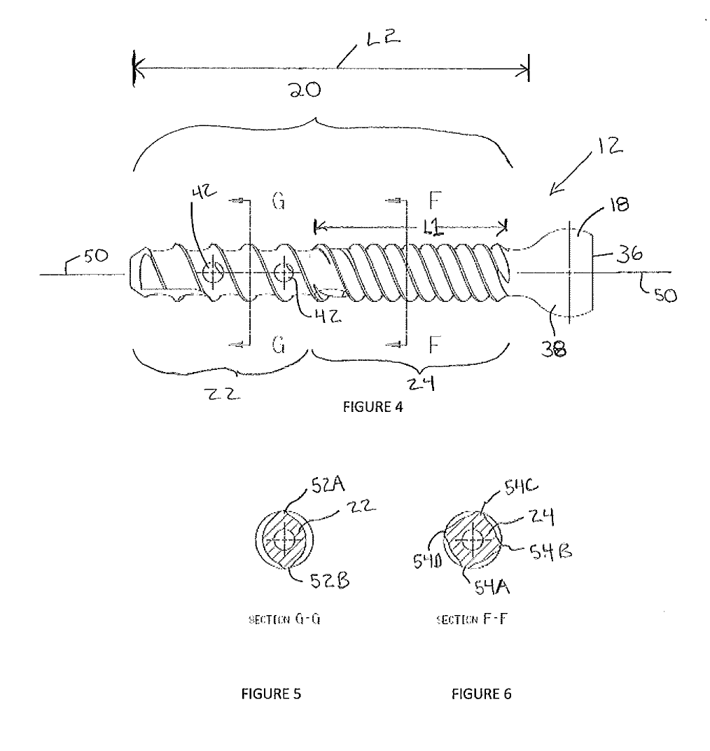

Continuing to refer to FIGS. 1-3 and also referring to FIG. 4, the proximal head 16 of the bone anchor 12 in the exemplary embodiment is generally in the shape of a truncated sphere having a planar proximal surface 36 and an approximately spherically shaped distal surface 38. The exemplary bone anchor assembly is a polyaxial bone screw designed for posterior implantation in the pedicle or lateral mass of a vertebra. In this regards, the proximal head 18 of the bone anchor 12 engages the distal end 32 of the receiver member 14 in a ball and socket like arrangement in which the proximal head 18, and thus the distal shaft 20, can pivot relative to the receiver member 14. The distal surface 38 of the proximal head 18 of the bone anchor 12 and the mating surface of the within the distal end 32 of the receiver member 14 may have any shape that facilitates this ball and socket like arrangement, including, for example, spherical (as illustrated), toroidal, conical, frustoconical, and any combinations of these shapes.

The distal shaft 20 of the bone anchor 12 may be cannulated, having a central passage or cannula 40 extending the length of the bone anchor 12 to facilitate delivery of the bone anchor 12 over a guide wire in, for example, minimally invasive procedures. The distal shaft 20 may also include one or more side wall openings 42 or fenestrations that communicate with the cannula 40 to permit bone in-growth or to permit the dispensing of bone cement or other materials through the bone anchor 10. The side wall openings 42 extend radially from the cannula 40 through the side wall of the distal shaft 20. Exemplary systems for delivering bone cement to the bone anchor assembly 10 and alternative bone anchor configurations for facilitating cement delivery are described in U.S. Patent Application Publication No. 2010/0114174, which is hereby incorporated herein by reference. The distal shaft 20 of the bone anchor 12 may also be coated with materials to permit bone growth, such as, for example, hydroxyl apatite, and the bone anchor assembly 10 may be coated all or in-part with anti-infective materials, such as, for example, tryclosan.

Continuing to refer to FIGS. 1-3, the proximal end 26 of the receiver member 14 of the exemplary bone anchor assembly 10 includes a pair of spaced apart arms 28A, 28B defining the U-shaped recess 30 therebetween for receiving a spinal fixation element. The distal end 32 of the receiver member 14 is generally cylindrical in shape and includes distal end surface 34 which is generally annular in shape defining a circular opening through which at least a portion of the bone anchor 12 extends. For example, the distal shaft 20 of the bone anchor 12 may extend through the opening. Each arm 28A, 28B of the proximal end 26 of the receiver member 14 extends from the distal end 32 of the receiver member 14 to a free end. The outer surface of each arm 28A, 28B may include a feature, such as a recess, dimple, notch, projection, or the like, to facilitate connection of the receiver member 14 and, thus, the bone anchor assembly 10, to instruments. In the exemplary embodiment, for example, the outer surface of each arm 28A, 28B includes an arcuate groove 44A, 44BA at the respective free end of the arms. Such grooves are described in more detail in U.S. Pat. No. 7,179,261, which is incorporated herein by reference.

The proximal end 26 of the receiving member 14 may be configured to receive a closure mechanism, such as internal set screw (closure mechanism 16) or an external cap or nut. For example, the interior surface of each arm 28A, 28B may include a feature, such as a recess, dimple, notch, projection, thread or the like, to facilitate connection of the closure mechanism 16 to the receiver member 14. In the exemplary embodiment, for example, the interior surface of each arm 28A, 28B includes an internal thread 46 on the interior surface of each arm 28A, 28B for engaging the closure mechanism 16. In the exemplary embodiment, the thread starts at the free, proximal end and extends distally along at least a portion of the length of the arms 28A, 28B.

The closure mechanism 16 in the exemplary embodiment is an internal set screw having an external thread that engages the internal thread of the receiver member to capture a spinal fixation element within the recess 30 of the receiver member and, when fully tightened, to fix the spinal fixation element relative to the receiver member 14. Alternatively, the closure mechanism may be dual closure mechanism having an inner and an outer set screw, such as, for example, the Expedium Dual Innie Polyaxial Screw available from DePuy Spine, Inc. of Raynham, Mass. In addition, the closure mechanism may be a non-threaded twist in cap, such as, for example, the Monarch Typhoon Cap available from DePuy Spine, Inc. of Raynham, Mass., and described in U.S. Pat. No. 6,755,829, incorporated herein by reference.

The exemplary bone anchor assembly 10 may be used with a spinal fixation element such as a rigid spinal rod. The spinal rod may be constructed titanium, titanium alloys, stainless steel, cobalt chrome, PEEK, or other materials suitable for rigid fixation. Alternatively, the spinal fixation element may be a dynamic stabilization member that allows controlled mobility between the instrumented vertebrae.

The exemplary bone anchor assembly is a rigid polyaxial screw in which the bone anchor 12 is fixed, rather than mobile, when the spinal fixation element is fixed to the receiver member 14 of the bone anchor assembly. The spinal fixation element may either directly contact the proximal head 18 of the bone anchor 12 or may contact an intermediate element, e.g., a compression member 100, interposed between the spinal fixation element and the proximal head 18 of the bone anchor 12 to compress the distal outer surface of the proximal head 18 into direct, fixed engagement with the distal inner surface of the receiver member 18 when the spinal fixation element is fixed to the receiver member 16 of the bone anchor assembly by the closure mechanism. In alternative embodiments, the bone anchor assembly may be a mobile screw in which the proximal head 18 of the bone anchor 12 can move relative to the receiver member 14 when the spinal fixation element is fixed to the receiver member 14. An exemplary mobile polyaxial screw is described is U.S. patent application Ser. No. 12/580,777, filed Oct. 16, 2009, which is hereby incorporated herein by reference. Alternatively, the bone anchor assembly may be a monoaxial screw, a favored angle screw or a uniplanar screw.

The threaded distal section 22 and the threaded proximal section 24 of the distal shaft of the bone anchor 12 may be configured to increase fixation of the bone anchor assembly 10 in bone. For a bone anchor assembly designed to be implanted through the pedicle of a vertebra, for example, the threaded distal section 22 may be configured to engage the cancellous bone in the anterior vertebral body of the vertebra and the threaded proximal section 24 may be configured to engage the cortical bone of the pedicle of the vertebra. In particular, the threaded distal section 22 may have a pitch that is greater than (i.e., more coarse) the pitch of the proximal section 24. To facilitate insertion of the bone anchor 12 into the vertebra and prevent stripping of the pedicle wall, the distal shaft 20, both the threaded distal section 22 and threaded proximal section 24, can have a constant thread lead. The lead of a thread is the distance the distal shaft 20 travels in a direction parallel to the longitudinal axis 50 of the shaft when the distal shaft 20 is rotated one turn) (360°. The lead of a thread is equal to the number of thread starts multiplied by the pitch of the thread. As the threaded distal section 22 and the threaded proximal section 24 have different pitches, the threaded distal section 22 and the threaded proximal section 24 must have a different number of thread starts in order to have a constant or equal lead. In the exemplary polyaxial bone anchor assembly 10, for example, the lead of the distal shaft 20 is 6 mm, the pitch of distal threaded section 22 is 3 mm and the distal threaded section 22 has two thread starts (i.e., the distal threaded section 22 is dual threaded) and the pitch of proximal threaded section 24 is 1.5 mm and the proximal threaded section 24 has four thread starts (i.e., the proximal threaded section 24 is quad threaded). FIG. 5 is a cross section of the distal threaded section 22 and illustrates the two thread crests 52A and 52B of the dual thread of the distal threaded section 22. FIG. 6 is a cross section of the proximal threaded section 24 and illustrates the four thread crests 54A-54D of the quad thread of the proximal threaded section 24. Table 1 provides a summary for the exemplary bone anchor assembly 10:

TABLE 1

| | Pitch | Starts | Lead |
|---|---|---|---|
| Distal Threaded Section 22 | 3 mm | 2 | 6 mm |
| Proximal Threaded Section 24 | 1.5 mm | 4 | 6 mm |

The lead of the threaded distal section 22 and the threaded proximal section 24 can vary depending on, for example, the type of bone anchor assembly (e.g., polyaxial, monoaxial, uniplanar) and the vertebra or other bone in which the assembly is to be implanted. For polyaxial bone anchors designed to be inserted through the pedicle of a lumbar or thoracic vertebra, for example, the lead may be from 4 mm to 8 mm and the pitch of the distal threaded section 22 may be from 2 mm to 4 mm, and the pitch of the proximal threaded section 24 may be from 1 mm to 3 mm. In monoaxial screws, for example, the lead may be 2 mm to 4 mm.

The axial length (i.e., the length in a direction parallel to the longitudinal axis 50) of the proximal threaded section 24 of the distal shaft 20 can vary depending on the vertebra or other bone in which the assembly is to be implanted and may be selected to correspond to the length of bone the proximal threaded section 24 will engage. For bone anchors designed to be inserted through the pedicle of a lumbar or thoracic vertebra, the axial length of the proximal threaded section 24 may be selected to approximate the length of the pedicle including the distance from the posterior surface of the vertebra through the pedicle to the junction of the pedicle and the anterior vertebral body of the vertebra. In such bone anchors, the axial length L1 of the proximal threaded section 24 may be between 14 mm and 26 mm and preferably is 20 mm. The axial length of the distal shaft 20 may also vary depending on the bone in which the bone anchor 12 is to be inserted. For bone anchors designed to be inserted through the pedicle of a lumbar or thoracic vertebra, the axial length L2 of the distal shaft 20 may be between 20 mm and 100 mm. For bone anchors designed to be inserted through the iliac, the axial length L2 of the distal shaft 20 may be between 60 mm and 150 mm.

The major diameter and the minor diameter of the distal threaded section 22 and the proximal threaded section 24 may be selected based on the bone in which the bone anchor 12 is to be inserted. For bone anchors designed to be inserted through the pedicle of a lumbar or thoracic vertebra (such as the exemplary bone anchor 12), for example, the major diameter of the distal threaded section 22 and the proximal threaded section 24 may be between 4 mm and 10 mm. In the exemplary embodiment, the major diameter of the distal threaded section 22 and the major diameter of the proximal threaded section 24 are equal and constant over the axial length of the distal threaded section 22 and the proximal threaded section 24. In the exemplary embodiment, the minor diameter of the proximal threaded section 24 is greater than the minor diameter of the distal threaded section 22. The increased minor diameter of the proximal threaded section 24 provides reduced thread depth for the proximal threaded section 24 which increases bone purchase by compressing the bone of the pedicle of the vertebra. The minor diameter of the distal threaded section 22 is constant over the axial length of the distal threaded section 22 and the minor diameter of the proximal threaded section 24 is constant over the axial length of the proximal threaded section 24. The minor diameter may increase step wise or gradually from the distal threaded section 22 to the proximal threaded section 24. Table 2 provides exemplary major and minor diameters for the distal threaded section 22 and proximal threaded section 24.

TABLE 2

| | | | Minor |
|---|---|---|---|
| | Distal Threaded | Proximal Threaded | Diameter |
| Major | Section Minor | Section Minor Diameter | Increase |
| Diameter (mm) | Diameter (mm) | (mm) | (mm) |
| 4.425 | 3.075 | 3.425 | 0.35 |
| 4.9 | 3.66 | 4.03 | 0.37 |
| 5.89 | 4.06 | 4.31 | 0.25 |
| 6.85 | 4.47 | 5 | 0.53 |
| 7.85 | 5.05 | 6 | 0.95 |
| 8.85 | 6.05 | 7 | 0.95 |
| 9.85 | 7.05 | 8 | 0.95 |
| 10.85 | 8.05 | 9 | 0.95 |
| 11.85 | 9.05 | 10 | 0.95 |

In alternative embodiments, the minor diameter of the distal threaded section 22 and the minor diameter of the proximal threaded section 24 may be equal and constant over the axial length of the distal threaded section 22 and the minor diameter of the proximal threaded section 24.

In alternative embodiments, the major diameter of the proximal threaded section 24 may be greater than the major diameter of the distal threaded section 22. The major diameter may increase step wise or gradually from the distal threaded section 22 to the proximal threaded section 24.

While the devices and methods of the present invention have been particularly shown and described with reference to the exemplary embodiments thereof, those of ordinary skill in the art will understand that various changes may be made in the form and details herein without departing from the spirit and scope of the present invention. Those of ordinary skill in the art will recognize or be able to ascertain many equivalents to the exemplary embodiments described specifically herein by using no more than routine experimentation. Such equivalents are intended to be encompassed by the scope of the present invention and the appended claims.

1. A bone anchor assembly comprising:
a bone anchor having a proximal head and a distal shaft configured to engage bone, the distal shaft including a distal threaded section and a proximal threaded section, the distal threaded section having a first pitch and a first number of thread starts, the proximal threaded section having a second pitch less than the first pitch and a second number of thread starts greater than the first number of thread starts, the distal threaded section and the proximal threaded section having a constant lead,
a receiver member for receiving a spinal fixation element to be coupled to the bone anchor, the receiver member having
a proximal end having a pair of spaced apart arms defining a recess therebetween,
a distal end having a distal end surface defining opening through which at least a portion of the bone anchor extends,
a closure mechanism positionable between and engaging the arms to capture a spinal fixation element within the receiver member and fix the spinal fixation element with respect to the receiver member.

2. The bone anchor assembly of claim 1, wherein the first number of thread starts is 2 and the second number of thread starts is 4.

3. The bone anchor assembly of claim 2, wherein first pitch is 6 mm and the second pitch is 1.4 mm and the constant lead is 6 mm.

4. The bone anchor assembly of claim 1, wherein the bone anchor includes a central passage extending from the proximal head through the distal shaft.

5. The bone anchor assembly of claim 4, wherein the shaft includes a plurality of side wall openings that communicate with the central passage.

6. The bone anchor assembly of claim 5, wherein the side wall openings extend radially from the central passage through a side wall of the distal shaft.

7. The bone anchor assembly of claim 1, wherein the distal threaded section has a major diameter and the proximal threaded section has a major diameter, wherein the major diameter of the distal threaded section is equal to the major diameter of the proximal threaded section.

8. The bone anchor assembly of claim 7, wherein the distal threaded section has an axial length and wherein the major diameter of the distal threaded section is constant over axial length of the distal threaded section.

9. The bone anchor assembly of claim 8, wherein the proximal threaded section has an axial length and wherein the major diameter of the proximal threaded section is constant over axial length of the proximal threaded section.

10. The bone anchor assembly of claim 9, wherein the distal threaded section has a minor diameter and the proximal threaded section has a minor diameter, wherein the minor diameter of the proximal threaded section is greater than the minor diameter of the distal threaded section.

11. The bone anchor assembly of claim 10, wherein the distal threaded section has an axial length and wherein the minor diameter of the distal threaded section is constant over axial length of the distal threaded section.

12. The bone anchor assembly of claim 11, wherein the proximal threaded section has an axial length and wherein the minor diameter of the proximal threaded section is constant over axial length of the proximal threaded section.

13. The bone anchor assembly of claim 9, wherein the distal threaded section has a minor diameter and the proximal threaded section has a minor diameter, wherein the minor diameter of the proximal threaded section is equal to the minor diameter of the distal threaded section.

14. The bone anchor assembly of claim 1, wherein the distal threaded section has a major diameter and the proximal threaded section has a major diameter, wherein the major diameter of the proximal threaded section is greater than the major diameter of the distal threaded section.

15. The bone anchor assembly of claim 1, wherein the proximal threaded section has an axial length of between 14 mm and 26 mm.
